(19) United States
(12) Patent Application Publication
(10) Pub. No.: US 2017/0273713 A1
(43) Pub. Date: Sep. 28, 2017

(54) INTRAVENOUS ACCESS ASSIST DEVICE

(71) Applicants: Amit Shah, North Potomac, MD (US); Curt Kothera, Rockville, MD (US)

(72) Inventors: Amit Shah, North Potomac, MD (US); Curt Kothera, Rockville, MD (US)

(21) Appl. No.: 15/612,707

(22) Filed: Jun. 2, 2017

Related U.S. Application Data
(60) Provisional application No. 62/345,593, filed on Jun. 3, 2016.

(30) Foreign Application Priority Data
Jan. 21, 2016 (US) PCT/US16/14250

(51) Int. Cl. A61B 17/34; A61M 25/06

(52) U.S. Cl. CPC A61B 17/3403; A61M 25/0612; A61M 5/427

(57) ABSTRACT

The present invention is a simple-to-use IV placement assist device with a base, finger interfaces extending from the base, and mechanical means to engage a needle hub. A catheter advancer is slidably integrated with the base to guide advancement of a catheter linearly along the insertion plane defined by the orientation of the base. In addition, a stabilizer component comprising a traction pad and means of constraining unintended movements of the needle and catheter, which is placed on the patient's skin, may be used along with the base and catheter advancer. The IV placement assist device stabilizes and guides the critical needle insertion and catheter advancement steps of a complication-prone and very common procedure.

### CROSS-REFERENCE TO RELATED APPLICATIONS

The present application derives priority from U.S. Provisional Patent Application 62/345,593 filed 3 Jun. 2016, and derives priority from International Application PCT/US16/14250 filed 21 Jan. 2016, which in turn derived priority from provisional application No. 62/106,403 filed 22 Jan. 2015.

### BACKGROUND OF THE INVENTION

Field of the Invention

The present invention relates to a device for assisting a user in gaining vascular access of a patient.

Description of the Background

Venous access is among the critical first steps in caring for patients in the pre-hospital, emergency department/trauma center, and in-hospital environments. Fluid therapy and medication delivery rely on adequate intravenous (IV) access and, accordingly, 60-90% of hospitalized patients require a peripheral IV during their hospital stay. Over 300 million peripheral IV's are sold yearly in the US and over 1 billion units are sold worldwide. However, in 12-26% of adult patients and 24-54% of pediatric patients, the first attempt at catheter insertion fails, requiring additional, painful attempts. Moreover, blood vessel trauma resulting from failed insertion attempts increases the risk of subsequent catheter failure, with failure defined as catheter removal before the end of its intended dwell time or before the CDC recommended 72-96 hour dwell time limit. Peripheral IV's fail at a rate of 35-50% due to painful processes such as inflammation (phlebitis); fluid or medication leakage into surrounding tissue (infiltration); dislodgment; mechanical failure (e.g., occlusion); and site or bloodstream infection. Unsuccessful IV insertion attempts and IV failures are expensive in terms of direct equipment costs; provider time; necessitating more invasive venous access procedures; management of complications; additional hospital days; and, of course, the patient's pain and dissatisfaction.

Providers with high levels of training and experience have a significantly higher first pass success rate and lower incidence of ultimate IV failure, both of which directly reduce the pain experienced by the patient. Of course, training and experience are, by their very nature, time-intensive and otherwise expensive to acquire. An innovative device that enables novices to mimic the fluid expert approach could be of great value, provided that it is rigorously designed to meet patient, disease process, user, environmental, size, weight, and cost requirements.

The past several years has seen innovation relevant to IV placement. Examples include ultrasound and near-infrared technologies to aid vessel identification; antibiotic-impregnated and other cleansing approaches to tubing, connectors and dressings; and novel approaches to catheter stabilization. However, little has been done to simplify the often difficult task of actual IV insertion. In fact, novel technologies often assume a baseline level of competency with IV placement—an assumption that is not supported by the literature cited above, nor recent reports of >20% nursing turnover and high nursing vacancy rates in emergency settings. Emergency Medical Services (EMS) providers also turn over frequently. Functionally, high turnover rates in emergency settings equate to less experienced providers attempting IV placement on patients who are often the most difficult to access (e.g., due to dehydration) and in the most immediate need. Again, a device that simplifies the most difficult aspects of IV insertion could be of value.

Several of the steps followed for IV insertion require precision and a steady hand to prevent the needle from going all the way through the vein. Senior nurses, and the literature, counsel new nurses to pay special attention to these steps, especially in patients who have difficult-to-access veins due to dehydration, excess or thin skin, scarring, obesity, and edema, among other conditions. As described in primers on IV placement written by nurse educators, the most common errors after appropriate target vessel identification and tourniquet placement are related to vein stabilization, angle of approach, and IV assembly advancement after a flash of blood is visualized, offering an opportunity for innovation.

### SUMMARY OF THE INVENTION

It is, therefore, an object of the present invention to provide an easy-to-use IV placement assist device that is designed to pull the skin taut and stabilize the vein, enhance catheter advancement, add needle/catheter stability, and decrease reliance on freehand approximation of the approach angle.

It is another object to provide an IV placement assist device that facilitates small movements of the needle/catheter assembly or either part individually, such as with advancement of the cannula over the needle; and prevent unwanted needle movement—all without interrupting the flow of the IV start procedure or reducing tactile feedback.

It is an overarching object to provide an IV placement assist device with the foregoing qualities that facilitates a higher first attempt IV placement success rate and decreased vessel trauma and, as a result, decreased IV failures, increased patient comfort and satisfaction, and decreased cost.

In accordance with the foregoing objects, the invention disclosed herein is a low-cost and simple-to-use IV placement assist device with a base, finger interface extending from the base, and mechanical means to engage a needle hub. A catheter advancer is slidably integrated with the base to guide advancement of a catheter linearly along the insertion plane defined by the orientation of the base. In addition, a stabilizer component comprising a traction pad and means of constraining movement of the needle and catheter, which is placed on the patient's skin surface, may be used along with the base and catheter advancer to assist the user in placing an intravenous catheter.

The foregoing components combine to form an integrated assistive device of the present invention. The result is an easy-to-use, handheld device that can stabilize and guide the critical needle insertion and catheter advancement steps of a complication-prone and very common procedure. The base serves as a stabilizing component that emanates from the user's hand, designed to function as an extension of his/her fingers. This component provides the user with means of gripping and controlling the typically small needle hub, as well as providing clear view of the flash chamber contained within the needle hub. Engagement with the needle hub is tight and secure in a manner that readily transmits forces acting on the needle, thereby maintaining tactile feedback to the user. The base also may have guide tracks along which the catheter advancer component slides, thereby providing linear and non-twisting insertion guidance of the catheter about the needle. The catheter advancer component slidably integrates with the base component and may come completely and freely apart from the base during proper use of the device during the catheter advancement step of the IV insertion procedure. Engagement between the catheter advancer and the catheter is specifically not tight and secure, so the component can be freely removed from the inserted catheter without any possibility of dislodging the catheter. The stabilizer component provides means of facilitating skin traction and preventing unintended motions of the needle or catheter tip during alignment, approach, and insertion steps of the procedure. In this device, the angle of approach is not specifically controlled, though use of all device components encourages the user to approach and insert at the preferred low angle. For example, the tip and traction pad of the stabilizer may be positioned apart from each other by a distance that, when holding the base over the user's fingers that are in contact with the traction pad, creates a desired approach and initial needle insertion angle (approx. 15-30 deg).

Key benefits of the intravenous access assist device are as follows:
The recreated expert approach will increase first-pass success rates for peripheral IV placement, avoiding the pain of multiple IV sticks.
First-pass success due to device use will, in turn, decrease painful complications downstream.
Procedural simplification will give novice users greater confidence.
Low-cost, unpowered, and disposable device, the intravenous access assist device will have broad applicability.
Adaptability to a variety of manufacturers' IV needles will facilitate technology transition and adoption.

### BRIEF DESCRIPTION OF THE DRAWINGS

Other objects, features, and advantages of the present invention will become more apparent from the following detailed description of the preferred embodiment and certain modifications thereof when taken together with the accompanying drawings in which:

FIG. 1 is a top perspective illustration of the intravenous access assist device with a catheter and needle seated therein.

FIG. 2 is a top perspective illustration of a catheter advancer integrated with a base in the starting position with a needle and catheter seated therein.

FIG. 3 is a top perspective illustration of a catheter advancer integrated with a base in an advanced position with a needle and catheter seated therein.

FIG. 4 is a top perspective illustration of a base component of the intravenous access assist device.

FIG. 5 is a bottom perspective illustration of a base component of the intravenous access assist device.

FIG. 6 is a top perspective illustration of a catheter advancer component of the intravenous access assist device.

FIG. 7 is a bottom perspective illustration of a catheter advancer component of the intravenous access assist device.

FIG. 8 is a top perspective illustration of a stabilizer component of the intravenous access assist device.

FIG. 9 is a bottom perspective illustration of a stabilizer component of the intravenous access assist device.

### DETAILED DESCRIPTION OF THE PREFERRED EMBODIMENT

The present invention is an IV placement assist device for assisting in the manual placement of an IV catheter without interrupting the flow of the IV start procedure.

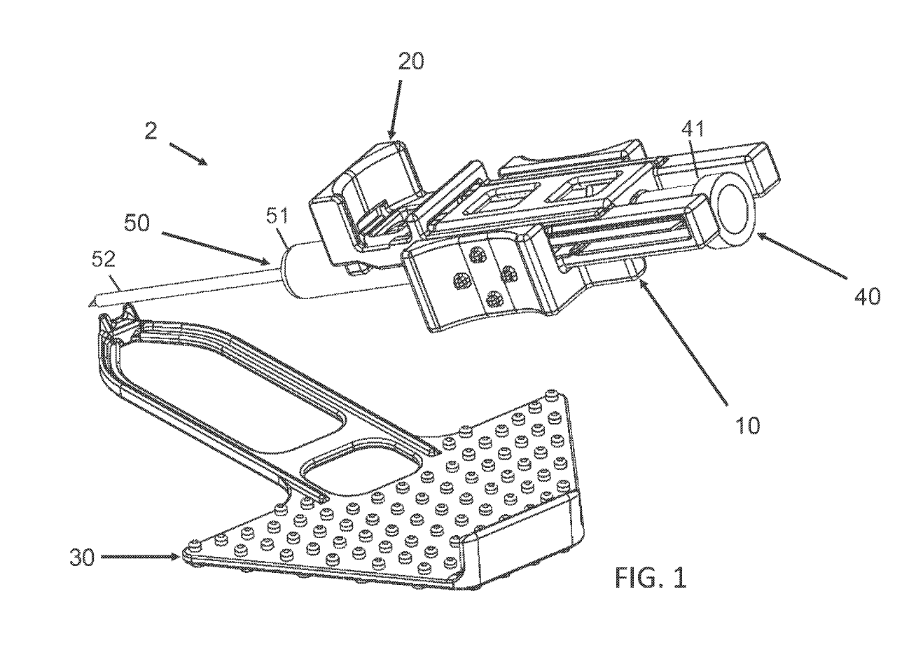

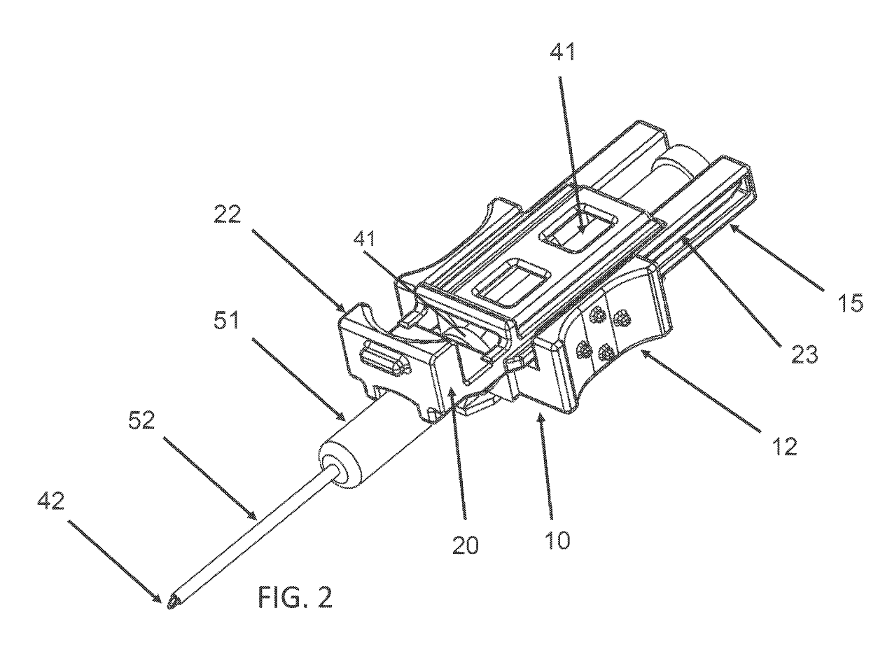

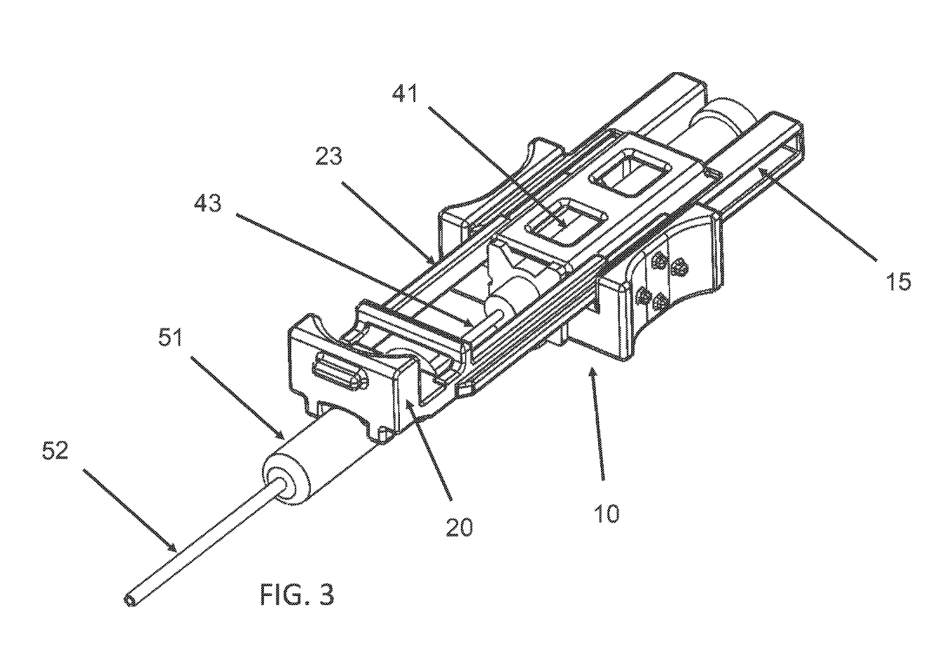

With reference to FIG. 1, the IV placement assist device 2 generally comprises a base 10, a catheter advancer 20, and a stabilizer 30. Given a conventional needle 40 carrying a deployable catheter 50, the base 10 securely engages needle 40 at its hub 41 and the catheter advancer 20 lightly engages catheter 50 at its hub 51. An alternate top perspective view without stabilizer 30 is shown in FIG. 2 when the catheter advancer 20 is in its starting position (not yet advanced). Here the catheter hub 51 is still in contact with the needle hub 41 and the needle tip 42 is visibly extending through the distal tip of catheter sleeve 52. FIG. 3 shows the same assembly of FIG. 2 when the catheter 50 has been advanced somewhat and a section of the needle shaft 43 is visible proximal to advanced catheter hub 51 through the catheter advancer 20 (between two catheter advancer legs 23).

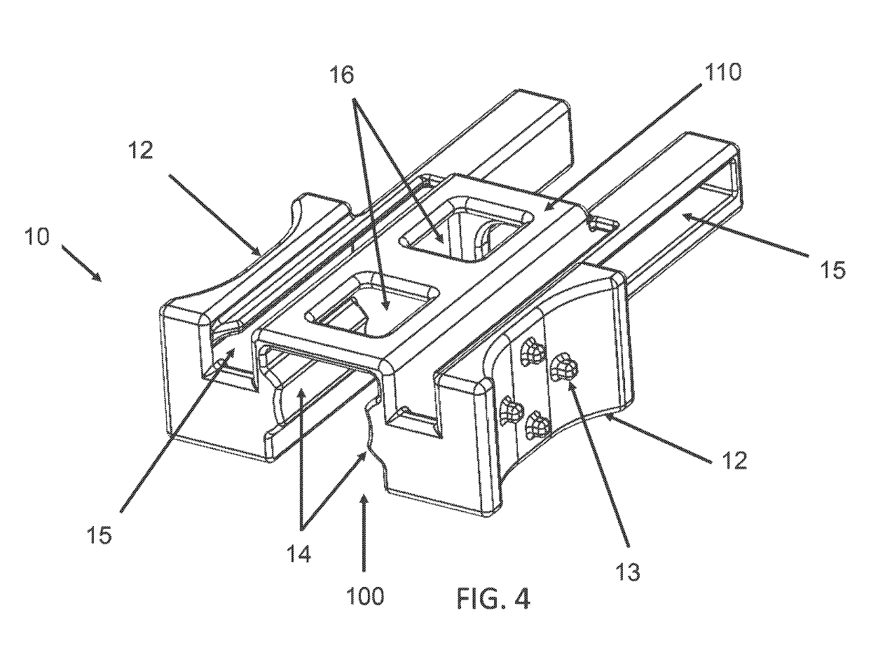

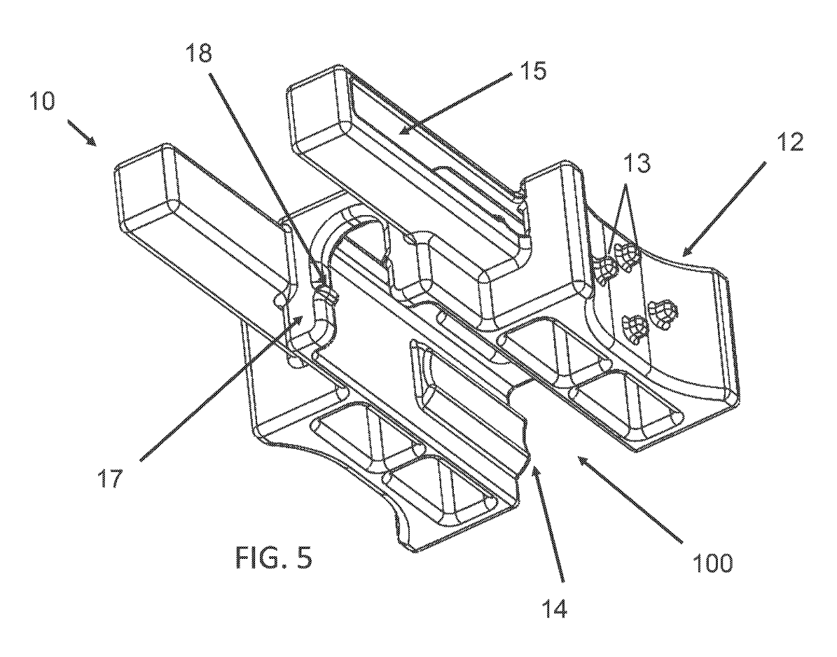

In reference to FIGS. 4-5, Base 10 preferably has finger interfaces 12 oriented on the lateral faces of base 20 such that the user can hold base 20 with a squeeze grip between the thumb and middle finger. Finger interfaces 12 also preferably provide a much wider grip such that the tips of the thumb and middle finger are spaced wide enough apart to clearly see between them, even for a gloved user with large fingers. To facilitate gripping, finger interfaces 12 are preferably concave and may contain additional features such as protrusions 13 for tactile feedback. Other patterned protrusions, indentions, and textures may also be used within the same invention.

As seen in FIG. 2 base 10 is intended to mechanically engage the hub 41 of a needle 40, preferably with a snap fit. As seen in FIG. 4 there is a channel 100, preferably in the bottom of base 10 through which the needle hub 41 is transversely inserted. As seen in FIG. 5 the channel 100 preferably has lateral securement features 14 that hold the needle hub 41, such as opposing walls with semi-cylindrical surfaces. Note that the needle-hub securement features 14 may include any such forms that generally produce the opposite shape of the needle being contained. A spanner 110 straddles the channel 100 at the top, and the spanner 110 is preferably of a thickness of resilient material that allows enough flex at features 14 for engaging the needle hub 41, yet remains stiff enough to securely hold the needle 40 and provide tactile feedback to the user. While lateral securement features 14 provide a lateral squeeze hold on the needle hub 41, base 10 may also incorporate a longitudinal constraint 17, herein shown as a yoke, which provides physical resistance to motion of the needle hub 41 within base 10. Longitudinal constraint 17 may also contain snap fit features 18, such as small protrusions.

To interface with catheter advancer 20, base 10 preferably has at least one sliding track 15 that constrains the advancement motion of the catheter advancer 20 along only the longitudinal axis of needle 40. In the preferred embodiment, there are two parallel tracks 15, with one on each lateral side of base 10 and their configuration also prevents twisting of catheter 50 about needle 40. To achieve a compact design, tracks 15 may be positioned between lateral securement features 14 and finger interfaces 12 on each side. This position also ensures a wider finger grip for clear visualization of the flash chamber, possibly (and often) within the needle hub 41, through at least one viewing window 16 through the spanner 110.

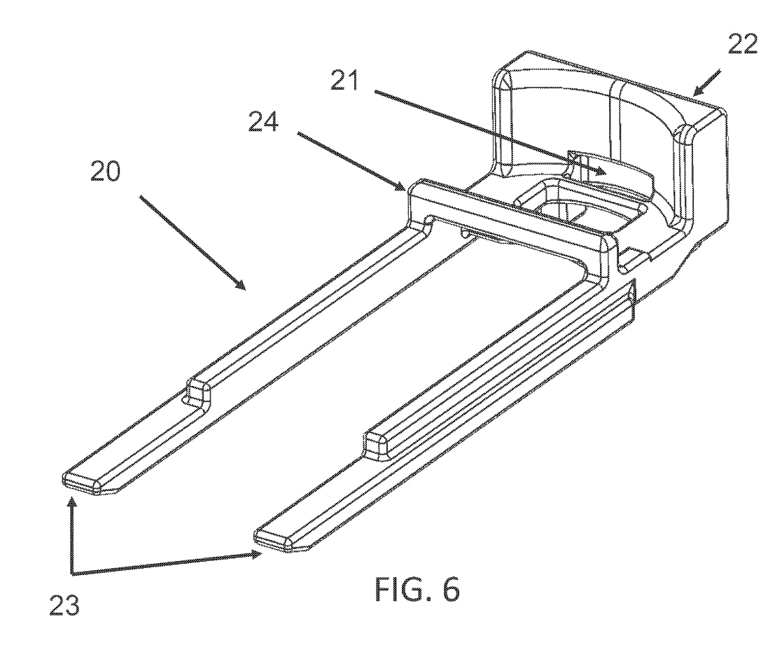

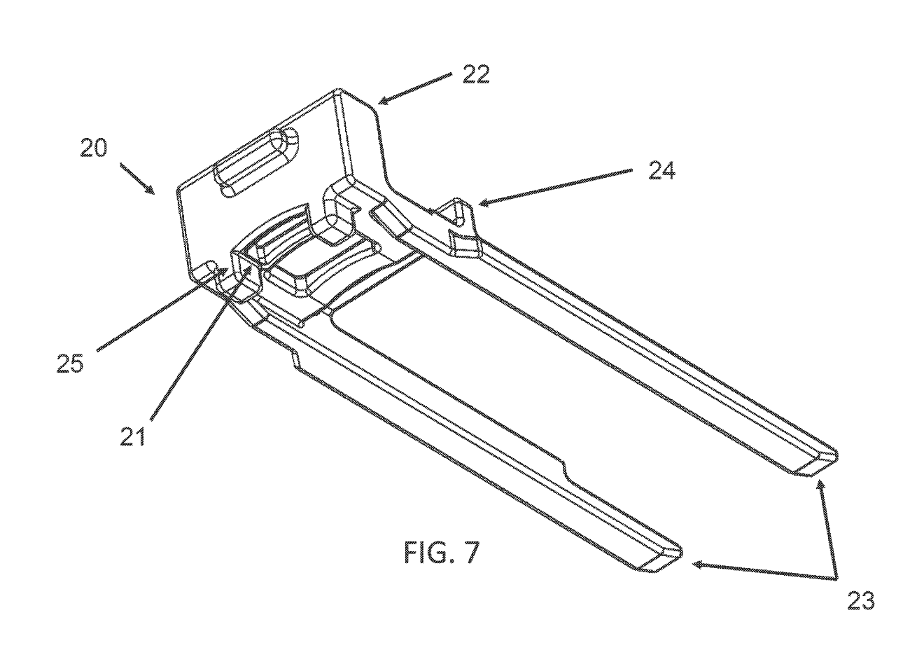

In reference to FIGS. 6-7, catheter advancer 20 is a fork-shaped member with two parallely-protruding rail guides 23 joined at one end by an upwardly angled finger interface 22. The finger interface 22 contains an engagement feature 21, such as a slot, which releasably engages a portion of catheter hub 51. By releasable engagement, it is meant that the catheter advancer 20 can be freely removed from the catheter hub 51 without the use of force or involvement of a second hand. Existing intravenous catheter systems, if at all, use a secure fit between the catheter hub and a feature that is intended to assist in its advancement. However, a secure fit here requires force, either longitudinal, rotational, or both, as well as assistance from a second hand, to disengage the catheter hub from the advancement feature. This extra force and procedural steps introduce opportunities to dislodge the inserted catheter 50, as well as added procedural time, and complications may result from dislodgement. It is an object of the present invention to provide free disengagement as described above.

Advancement of catheter 50 using catheter advancer 20 is facilitated through finger interface 22, which is shaped and configured as a right-angle tab to be pushed by the user's index finger. Finger interface 22 is preferably at least as wide and as tall as the tip of a small user's finger. Finger interface 22 is intended to be easy and comfortable to use by users with large, gloved fingers, so it is appropriately sized to accomplish this function. Finger interface 22 preferably extends vertically from rail guides 23, and perpendicular to the longitudinal axis of the needle 40 and catheter 50. The finger interface 22 may have some concavity to prevent slipping of the user's finger. Other textures and features may be added within the same invention. One embodiment of the invention may also incorporate a smaller finger interface 24 (see FIG. 7), proximal to the primary advancing interface 22. Smaller interface 24 may help the user with catheter retraction motions. Interface 24 is smaller than interface 22 to enable natural placement of a user's index finger between the two interfaces 22, 24 and enable their intended functions.

Catheter advancer 20 has at least one and preferably two rail guides 23 that slidably integrate with track(s) 15 of base 10. Rail guide 23, through slidable integration with track 15, is constrained to provide linear, non-twisting advancement of catheter hub 51 about needle shaft 43 when distal, longitudinal force is applied at finger interface 22. Preferably there are two rail guides 23, laterally spaced to fit outside the width of needle hub 41. Catheter advancer 20 may contain features to facilitate maintaining engagement with the catheter hub 51, while also enabling free disengagement thereof. These features may include at least one release member 25 and preferably two lateral members 25 as shown in FIG. 7. Note that the cross-sectional shape of the rail guides 23 is preferably L-shaped or T-shaped, though any other shape may also be used within the same invention, such as rectangular, circular, and triangular. During advancement of catheter 50, one embodiment of the present invention may have catheter advancer 20 slide completely out of base 10, and subsequently fall freely off of the inserted catheter 50.

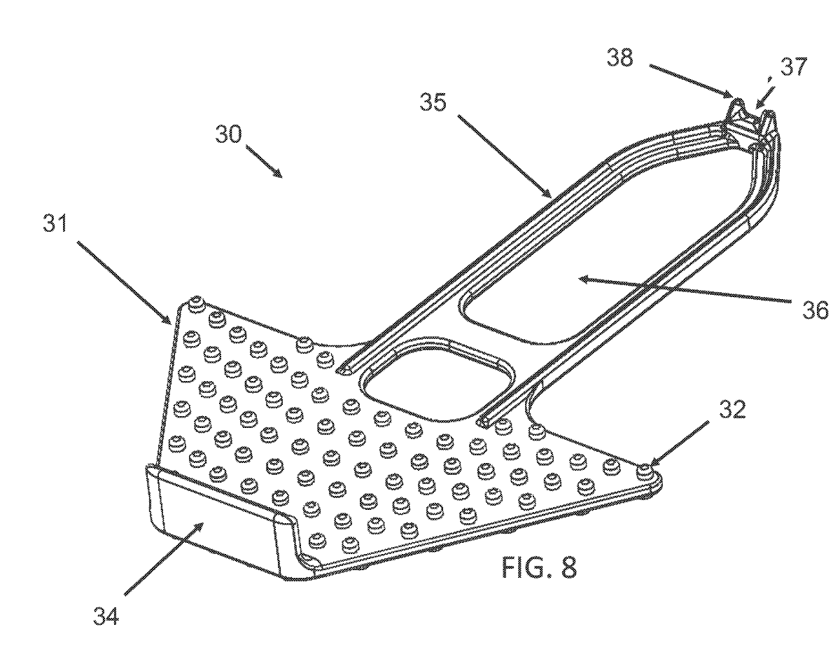

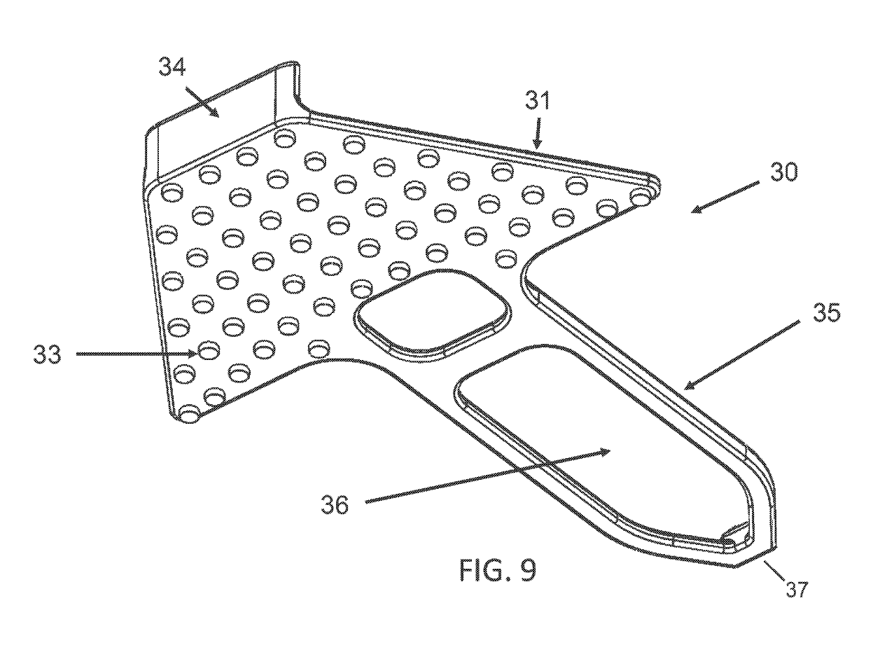

In reference to FIGS. 8-9, stabilizer 30 is of a relatively flat, laterally symmetric shape. The lateral symmetry enables ready use by both left- and right-handed users. Stabilizer 30 is configured to be anchored to a patient's skin surface by a traction pad 31 that is generally at least two finger-widths long in the longitudinal direction and at least three finger-widths wide. Traction pad 31 is relatively flexible such that it can conform to the patient's skin surface shape when downward pressure is applied by the user. The upper surface of traction pad 31 is preferably patterned with arrays of small protrusions 32 to promote grip between the user's fingers and stabilizer 30. Similarly, the lower surface of traction pad 31 is also preferably patterned with arrays of small protrusions 33 to promote grip between stabilizer 30 and the patient's skin. While cylindrical protrusions are pictured in the exemplary embodiment, any such protrusion, indentations, or surface textures may be applied within the same invention, and the same type of features need not be used for both the upper 32 and lower 33 sides. As an added measure of slip prevention between the user and stabilizer 30, a backstop 34 may extend upward near the proximal end.

Extending distally from traction pad 31 are legs 35, which are preferably spaced apart sufficiently wide to create window 36, through which the user can clearly visualize the target vessel in which catheter 50 will be inserted. Legs 35 come together at the distal end to form a tip 37, which provides a constraint on needle and catheter motion in one direction when the needle and catheter are rested on it. Tip 37 may also incorporate other features, such as upwardly protruding yoke 38, which provide additional stop limit constraints on needle 40 and catheter 50 motion at their respective tips. In the pictured preferred embodiment, needle tip 42 is constrained vertically and laterally when placed on stabilizer tip 37 between protrusions 38, such that the effect of any micro-manipulations at the user's hand are minimized or removed prior to and during insertion. By incorporating such a component that combines skin traction and needle tip stabilization, errors due to patient movement, especially in ambulatory environments, are minimized since the needle tip and patient skin are coupled and will move together.

In operation, the intravenous access assist device 2 is assembled and a needle 40 carrying a deployable catheter 50 is loaded therein such that the catheter advancer 20 lightly engages catheter 50 at its hub 51. The needle 40/catheter 50 combination is advanced by pushing the catheter advancer 20 for linear translation there along until base 10 securely engages needle 40 at its hub 41, at which point continued translation pushes the catheter 50 off needle 40.

It should now be apparent that the foregoing provides an easy-to-use IV placement assist device that is designed to pull the skin taut and stabilize the vein, and decrease reliance on freehand approximation of the approach angle, insertion of the needle, and advancement of the catheter. The device enables greater control in the needle/catheter assembly for approach and advancement, facilitates small movements of the needle/catheter or either part individually, and prevents unwanted movement, all without interrupting the flow of the IV start procedure. This facilitates more efficient IV placement and success rates, and decreases vessel trauma, IV failure, and patient discomfort.

Having now fully set forth the preferred embodiments and certain modifications of the concept underlying the present invention, various other embodiments as well as certain variations and modifications thereto may obviously occur to those skilled in the art upon becoming familiar with the underlying concept. It is to be understood, therefore, that the invention may be practiced otherwise than as specifically set forth herein.

We claim:

1. An intravenous access assist device for guiding insertion of a needle carrying a deployable catheter and for deploying said catheter from said needle, comprising:
a base having a finger grip for manipulating said base, and is mechanically engaged to a hub of said needle; and
an advancer slidably attached to said base for translation there along, said advancer having features enabling releasable engagement of hub of said catheter.

2. An intravenous access device of claim 1 wherein said features enabling releasable engagement freely disengage when said intravenous catheter advances beyond the tip of said needle.

3. The intravenous access device of claim 1, wherein said base is affixed to a hub of a needle.

4. The intravenous access device of claim 1, wherein said base is configured to be releasably coupled with said hub of said needle.

5. The intravenous access device of claim 1, wherein said base is comprised of an aperture for viewing said hub of a needle.

6. The intravenous access device of claim 5, wherein said aperture is located in the top of said base.

7. The intravenous access device of claim 1, wherein said advancer is linearly translatable along said longitudinal axis of said needle.

8. The intravenous access device of claim 1, wherein said finger grip comprises opposing concave notches configured to interface with a human finger.

9. The intravenous access device of claim 10, wherein said concave notches comprise surface texture to prevent finger slipping.

10. The intravenous access device of claim 10, wherein said concave notches comprise surface features for tactile feedback.

11. The intravenous access device of claim 1, wherein said advancer further comprises a first upwardly protruding finger tab.

12. The intravenous access device of claim 11, wherein said advancer further comprises a second upwardly protruding finger tab that is shorter than said first upwardly protruding finger tab.

13. The intravenous access device of claim 11, wherein said advancer finger tab comprises a concave notch.

14. The intravenous access device of claim 11, wherein said advancer finger tab is textured to prevent slipping.

15. The intravenous access device of claim 1, wherein said advancer comprises opposing legs slidably engaged to said base.

16. The intravenous access device of claim 1, wherein said advancer comprises a protruding collar for seating said catheter hub.

17. The intravenous access device of claim 1, wherein said base further comprises two parallel-spaced guide tracks on which said advancer slides.

18. The intravenous access device of claim 1, further comprising a stabilizer having a skin contacting surface and at least one traction pad.

19. The intravenous access device of claim 1, wherein said advancer slidably disconnects from said base.

20. The intravenous access device of claim 4, wherein said base has an upstanding collar for gripping said needle hub by a snap fit.

21. The intravenous access device of claim 18, wherein said stabilizer further comprises a pair of legs extending from said traction pad.

22. The intravenous access device of claim 18, wherein said traction pad comprises a plurality of bumps to reduce slipping.

23. The intravenous access device of claim 21, wherein said pair of legs converge to an upwardly protruding tip.

24. The intravenous access device of claim 17, wherein said guide tracks are linear.

25. The intravenous access device of claim 4, wherein said base comprises at least one collar.

26. The system of claim 23, wherein said upwardly protruding tip constrains motion of a needle or catheter in at least one direction.
